(19) United States
(12) Patent Application Publication
(10) Pub. No.: US 2008/0173558 A1
(43) Pub. Date: Jul. 24, 2008

(54) Fragrance-emanating nasal mask device and method

(76) Inventor: Contelanea Arvanitis, Alamo, CA (US)

Correspondence Address: SQUIRE, SANDERS & DEMPSEY L.L.P., 1 MARITIME PLAZA, SUITE 300, SAN FRANCISCO, CA, 94111, US

(21) Appl. No.: 11/656,691

(22) Filed: Jan. 22, 2007

(51) Int. Cl. A61M 15/02; B65D 69/00

(52) U.S. Cl. 206/232; 128/202.13

(57) ABSTRACT

The current invention relates to a nose mask for administering gas to a patient wherein a removable carrier within the mask emanates a pleasant fragrance, a method for constructing the mask and a kit for using the mask.

### FIELD

The current invention relates to a fragrance-emanating nasal mask and method of constructing same for use when delivering a gas to a patient. In particular, it relates to a nasal mask for dental use and most particularly for pediatric dental use.

### BACKGROUND

Health care professionals often desire to administer a gas to a patient over an extended period of time. A common means of accomplishing this task is to use a nasal mask which fits over and encloses the patient's nose and seals against the patient's face around the nose. Adult and adolescent patients sometimes complain about the odor of the mask itself and/or the odor of the gas being administered. Infants and children likewise may likewise find the odor of the mask or of the gas being delivered unpleasant and thereupon elect to breath through their mouths to avoid the odor and thereby defeating the purpose of the gas. More importantly infants and children often have difficulty comprehending the concept of breathing through the nose per se, which likewise can render administration of the gas using a nasal mask difficult if not impossible.

A partial solution to this situation has been the use of fragrances to conceal the odor of the nasal mask and gas being administered. The use of a patient-selected pleasant fragrance made the procedure being undergone more palatable for adults and adolescents and would encourage infants and young children to breath through their noses in order to be able to enjoy their selected fragrance. To date, two approaches have been employed for including a fragrance during administration of a gas to a patient. One is to impregnate the material of which a disposable nasal mask is constructed with a fragrance. The problems with this approach are that the practitioner must stock a fairly large quantity of disposable masks so as to provide the patient with a choice of fragrances. Further, nasal masks must either be sterilizable for reuse or for one-time use only. If sterilizable, in general the fragrance may be lost during the process, which often involves relatively high temperatures. If for one-time use, the pre-impregnated masks become both relatively costly and environmentally unfriendly. Even in the case of dental use, where a patient could retain the mask in his/her possession after use and bring it with them to future appointments, in a relatively short time the fragrance would dissipate from the mask, requiring replacement and/or the patient would forget to bring the mask with him/her to an appointment thereby necessitating the use of a new one.

The second approach to accomplishing the above task is to introduce the fragrance into the gas stream upstream from the nasal mask. An example of this can be seen in U.S. Pat. No. 6,842,218. While this approach is effective with new fragrance always being available, it requires specialized equipment and training to operate properly, making it an expensive alternative.

What would be desirable is a means of providing a fragrance during the administration of a gas to a patient that is effective, inexpensive and environmentally sound. The present invention provides the means for accomplishing this goal.

### SUMMARY

Thus, in one aspect the present invention relates to a nasal mask for delivering a gas to a patient, comprising a fragrance-emanating carrier removably coupled to an interior surface of the mask.

In an aspect of this invention, the fragrance-emanating carrier is impregnated with one or more fragrance(s).

In an aspect of this invention, at least one of the fragrances comprises an essential oil.

In an aspect of this invention, at least one of the fragrances comprises a synthetic fragrance.

In an aspect of this invention, the fragrance-emanating carrier comprises a fabric.

In an aspect of this invention, the fabric comprises cotton gauze.

In an aspect of this invention, the fragrance-emanating carrier comprises a polymer.

In an aspect of this invention, the polymer comprises a hydrogel.

In an aspect of this invention the patient is a medical patient.

In an aspect of this invention, the medical patient is a pediatric patient.

In an aspect of this invention, the patient is a dental patient.

In an aspect of this invention, the dental patient is a pediatric patient.

An aspect of this invention is a method of providing a fragrance during administration of a gas to a patient, comprising providing a nasal mask, providing a carrier, providing one or more fragrances, impregating the carrier with one or more fragrances, removably coupling the impregnated carrier to an interior surface of the nasal mask, placing the mask over the nares of the patient and administering the gas through the mask.

In an aspect of this invention in the above method the carrier comprises a fabric.

In an aspect of this invention in the above method the fabric comprises cotton gauze.

In an aspect of this invention in the above method impregnating the fabric comprises dipping the fabric in a solution or suspension of the fragrance in a suitable liquid.

In an aspect of this invention in the above method impregnating the fabric comprises spraying the fabric with a solution or suspension of the fragrance in a suitable liquid.

In an aspect of this invention in the above method impregnating the fabric comprises rubbing a surface of the carrier with a fragrance-containing semi-solid material.

In an aspect of this invention in the above method removably coupling the carrier to an interior surface of the nasal mask comprises placing the carrier dampened with the fragrance against the interior surface of the mask.

In an aspect of this invention in the above removably coupling the carrier to an interior surface of the nasal mask comprises placing the carrier in an open-mesh pouch that has been permanently adhered to the interior surface of the mask.

In an aspect of this invention in the above method removably coupling the carrier to an interior surface of the nasal mask comprises permanently adhering one member of a hook and loop fastener system to the interior surface of the nasal mask and permanently adhering the other member to the carrier and then bringing the two members into contact.

An aspect of this invention is a kit comprising a plurality of fragrances dissolved or suspended in a suitable liquid, each different fragrance being contained in a separate air-tight container, instructions for use of the kit and required FDA notices.

In an aspect of this invention, in the above kit the container is a metered delivery container.

In an aspect of this invention, in the above kit the metered delivery container comprises a nebulizer.

An aspect of this invention is a kit comprising a plurality of fragrance-emanating carriers, each carrier that emanates a different fragrance being contained in a separate air-tight container, instructions for use of the kit and required FDA notices.

In an aspect of this invention, in the above kit the air-tight container is resealable and contains a plurality of the fragrance-emanating carriers.

### DETAILED DESCRIPTION

The present invention provides a,construct and method for adding a fragrance when administrating a gas to a patient in need thereof through a nasal mask. The mask and method are intended for both medical and dental use; however, in a presently preferred embodiment it provides a means of delivering a fragrance during dental procedures when nitrous oxide, an effective mild sedative/anesthetic, is often administered to a patient to ease the patient's apprehensions and pain during the procedure. While the invention is useful for patients of any age, with older patients opting for fragrance-emanating masks simply to make the use of the mask more enjoyable and the procedure more palatable, it is particularly useful with younger pediatric patients. This is because it is often difficult to get very young patients to breath through their noses, which defeats the purpose of the nitrous oxide, which must be inhaled into the lungs to be effective. Providing an enjoyable fragrance automatically encourages nasal breathing in order for the child to partake of and enjoy what can be a personally selected favorite fragrance.

As used herein, a “nasal mask” refers to a device used to deliver a gas to a medical or dental patient through the nares or nasal openings. Such devices are common in the medical and dental professions and are very well known to those skilled in the medical and dental arts. Countless nasal mask designs are known, for example, without limitation, those depicted in U.S. Pat. Nos. 7,152,599, 7,044,130, 6,959,710, 6,923,181, 6,886,564, 6,871,649, 6,752,149, 6,729,333, 6,694,973, 6,662,803 and 6,651,663 and U.S. Design Pat. No. D519,206, D507,831, D506,827, D506,546, D502,261, D493,523 and D485,905, all of which are incorporated by reference as if fully set forth herein including all drawings. It is understood that the current invention may be employed with any of the foregoing nasal masks as well as any other nasal mask currently known or as may become known in the art in the future.

The gas that is administered using the nasal mask can be therapeutic, sedative, anesthetic, atmospheric or a controlled environment gas such as oxygen. It may be administered to a medical patient during a medical procedure or to a dental patient during a dental procedure. A presently preferred gas is nitrous oxide, a mild sedative/anesthetic which is often administered to patients, in particular dental patients, to ease the apprehension and pain that sometimes accompanies a dental procedure.

As used herein, a “carrier” refers to any substance or construct that is biocompatible and physiologically safe, particularly for use with young children and that can be impregnated with a fragrance. By “physiologically safe” is meant that the substance or construct causes no harm whatsoever to the health and well-being of a patient to whom a fragrance is being administered pursuant to this invention. A presently preferred carrier comprises a fabric which is capable of absorbing fragrances or liquids in which fragrances are dissolved or dispersed. The fabric may be natural, such as cotton or wool. In a presently preferred carrier embodiment of this invention the carrier is cotton gauze such as that used for surgical dressings and the like.

Another presently preferred carrier is a polymer that is capable of absorbing and releasing a fragrance such as, without limitation, a hydrogel which is capable of absorbing large quantities of water that can be infused with a selected fragrance.

While not absolutely necessary, it is presently preferred that the carrier be sterile prior to use.

The carrier may be of any size and shape that is capable of holding sufficient fragrance for the required time period to complete the procedure in which it is being used and of efficiently releasing the fragrance into the atmosphere.

By “fragrance-emanating carrier” is simply meant that once the carrier has been impregnated with a fragrance, it will release the fragrance into the atmosphere over time.

By “removably coupled” is meant that the carrier can be placed inside a nasal mask in such a manner that it either adheres to the wall of the mask simply by virtue of being moistened with the fragrance, i.e. by the well-known adhesive properties of water to a surface. Or the carrier may include a temporary, physiologically acceptable adhesive that will adhere to the interior of the mask. In the alternative, “removably coupled” means that the carrier can be placed into a carrier pouch that has been permanently adhered to the inside of the nasal mask, the carrier pouch being constructed of a sterilizable material so that the mask can be reused and having a mesh-like configuration so as to not impede the flow of the fragrance from the carrier into the mask. In yet another alternative, a hook and loop fastener system such as that commercially known as Velcro® may be employed. Either the hook or the loop member of the system can be fastened, either permanently or by means of a temporary adhesive, to the inside of the mask and the other member of the system can be fastened, likewise permanently or by means of a temporary adhesive, to the carrier. When the two are brought together, the carrier is held firmly in place by the hook and loop construct.

By “impregnated” is meant that the fragrance or a liquid containing the fragrance, when applied to a surface of the carrier, may either permeate into the interior or the carrier or may for the most part sit on the surface of the carrier or a combination thereof. The former would result when, to impregnate the carrier, the carrier is dipped into or sprayed with a solution or suspension of the fragrance in a suitable liquid. While any physiologically acceptable liquid may be suitable, for instance ethanol or an FDA-approved oil, a presently preferred liquid is water. Generally such a fragrance-containing liquid will saturate the carrier. On the other hand, if the fragrance is contained in a physiologically acceptable grease such as beeswax or petrolatum which is rubbed over the surface of the carrier, only a small amount may permeate into the interior of the carrier, the bulk of the material remaining in the grease and on the outer surface of the carrier.

As used herein, a “fragrance” has its ordinary meaning, e.g., “(a) sweet or delicate odor (such as fresh flowers, pine trees, or perfume or (b) something (as perfume) compounded to give off a sweet or pleasant odor.” Merriam-Webster On-line Dictionary.

As used herein, an “essential oil” refers to any member of a class of naturally-occurring oily substances that give plants their characteristic odors. Examples of essential oils include, without limitation, absinthe, angelica root, anise star, aniseed, mint bush, basil, bay, benzoin, bergamot, betel leaf, black current seed, camphor caraway, cardamom, cedarwood, celeryseed, chamomile, cinnamon, citral, clove, coffee, cognac, coriander, cumin, dill, eucalyptus, fennel, fir, frankincense, geranium, ginger, grapefruit, juniper, key lime, lavender, lemon, lime, menthol, nutmeg, orange, peppermint, pine, rose, sandalwood, savory, spearmint, spruce, tangerine, thyme, wintergreen and the like. Many other essential oils are known by those of ordinary skill in the art and all are within the scope of this invention. Of course, mixtures of two or more essential fragrances to produce a desired final fragrance are also within the scope of this invention.

As used herein, a “synthetic fragrance” refers to a fragrance that is synthetically compounded. It may comprise a single compound synthesized ab initio, a mixture of two or more such synthetic compounds, a mixture of essential oils or a mixture of one or more essential oils with one or more synthetically compounded fragrances. Examples of synthetic fragrances include, without limitation, allspice, apple pie, bay rum, bayberry, bitter almond, blueberry, bubblegum, butterscotch, chocolate, coconut cream, cucumber, daffodil, English lavender, French vanilla, fruit slices, grape, Hawaiian plumeria, honeydew melon, jasmine, lemon blossom, lilac, mango papaya, maple sugar, mountain laurel, ocean breeze, orange cinnamon, passion fruit, peachs & cream, pineapple, pomegranate, pumpkin, sandalwood, spiced rum, strawberry, sweet pea, sweet balsamic, tutti-fruitti, violet, watermelon and the like. Any such fragrance is likewise within the scope of this invention.

The fragrances herein may be applied to a carrier as-is, that is in their pure form, or they may be dissolved or diluted with a suitable liquid, where “suitable” refers to the liquid being environmentally and physiologically safe. As mentioned previously a presently preferred carrier is water in which a fragrance or combination of fragrances is dissolved or dispersed.

As used herein, a “patient” refers to a human being in need of a procedure, medical or dental, that the practitioner believes requires or at least would benefit from the administration of a gas using a nasal mask.

As used herein, “pediatric patient” has its normal meaning, i.e., infants, toddlers, children and adolescents but in particular refers to infants, toddler and children.

As used herein, a “kit” refers to an assembly of materials that are used in carrying out the method of the invention. A kit may also include instructions for the use of the enclosed material and any regulatory literature, such as FDA approvals and environmental statements as required by law or as desired by the kit provider.

Alternative means for accomplishing this invention may become apparent to those skilled in the art based on the disclosures herein. Any such alternative derived from this disclosure is within the scope of this invention.

What is claimed:

1. A nasal mask for delivering a gas to a patient, comprising a fragrance-emanating carrier removably coupled to an interior surface of the mask.

2. The nasal mask of claim 1, wherein the fragrance-emanating carrier is impregnated with one or more fragrance(s).

3. The nasal mask of claim 2, wherein at least one of the fragrances comprises an essential oil.

4. The nasal mask of claim 2, wherein at least one of the fragrances comprises a synthetic fragrance.

5. The nasal mask of claim 2, wherein the fragrance-emanating carrier comprises a fabric.

6. The nasal mask of claim 5, wherein the fabric comprises cotton gauze.

7. The nasal mask of claim 2, wherein the fragrance-emanating carrier comprises a polymer.

8. The nasal mask of claim 7, wherein the polymer comprises a hydrogel.

9. The nasal mask of claim 1, wherein the patient is a medical patient.

10. The nasal mask of claim 9, wherein the medical patient is a pediatric patient.

11. The nasal mask of claim 1, wherein the patient is a dental patient.

12. The nasal mask of claim 11, wherein the dental patient is a pediatric patient.

13. A method of providing a fragrance during administration of a gas to a patient, comprising:
providing a nasal mask;
providing a carrier;
providing one or more fragrances;
impregating the carrier with the one or more fragrances;
removably coupling the impregnated carrier to an interior surface of the nasal mask;
placing the mask over the nares of the patient; and,
administering the gas through the mask.

14. The method of claim 13, wherein the carrier comprises a fabric.

15. The method of claim 14, wherein the fabric comprises cofton gauze.

16. The method of claim 14, wherein impregnating the fabric comprises dipping the fabric in a solution or suspension of the fragrance in a suitable liquid.

17. The method of claim 14, wherein impregnating the fabric comprises spraying the fabric with a solution or suspension of the fragrance in a suitable liquid.

18. The method of claim 14, wherein impregnating the fabric comprises rubbing a surface of the carrier with a fragrance-containing semi-solid material.

19. The method of claim 13, wherein removably coupling the carrier to an interior surface of the nasal mask comprises placing the carrier dampened with the fragrance against the interior surface of the mask.

20. The method of claim 13, wherein removably coupling the carrier to an interior surface of the nasal mask comprises placing the carrier in an open-mesh pouch that has been permanently adhered to the interior surface of the mask.

21. The method of claim 13, wherein removably coupling the carrier to an interior surface of the nasal mask comprises permanently adhering one member of a hook and loop fastener system to the interior surface of the nasal mask and permanently adhering the other member to the carrier and then bringing the two members into contact.

22. A kit comprising:
a plurality of fragrances dissolved or suspended in a suitable liquid, each different fragrance being contained in a separate air-tight container.
instructions on using the kit; and,
any required FDA notices and approvals.

23. The kit of claim 22, wherein the container is a metered delivery container.

24. The kit of claim 23, wherein the metered delivery container comprises a nebulizer.

25. A kit comprising:
a plurality of fragrance-emanating carriers, each carrier that emanates a different fragrance being contained in a separate air-tight container;
instructions on using the kit;
any required FDA notices and approvals.

26. The kit of claim 25, wherein the air-tight container is resealable and contains a plurality of the fragrance-emanating carriers.
